(19) United States
(12) Patent Application Publication
(10) Pub. No.: US 2005/0119318 A1
(43) Pub. Date: Jun. 2, 2005

(54) Inhibitors of HCV replication

(76) Inventors: Thomas W. Hudyma, Durham, CT (US); Xiaofan Zheng, Cheshire, CT (US); Jeffrey Lee Romine, Meriden, CT (US)

Correspondence Address: STEPHEN B. DAVIS;BRISTOL-MYERS SQUIBB COMPANY, PATENT DEPARTMENT, P O BOX 4000, PRINCETON, NJ, 08543-4000, US

(21) Appl. No.: 10/974,588

(22) Filed: Oct. 27, 2004

Related U.S. Application Data
(60) Provisional application No. 60/516,338, filed on Oct. 31, 2003.

(51) Int. Cl. C07D 417/02; C07D 413/02; C07D 43/02; A61K 31/427; A61K 31/422; A61K 31/405

(52) U.S. Cl. 514/365; 514/381; 514/414; 514/419; 548/254; 548/181; 548/215; 548/465; 548/311.1; 514/374; 514/397

(57) ABSTRACT

Compounds having the structure of formula (I) 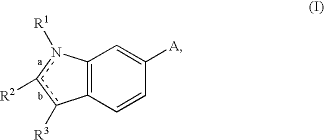 are disclosed. The compounds can inhibit hepatitis C virus (HCV) replication, and in particular the function of the HCV NS5B protein.

### CROSS-REFERENCE TO RELATED APPLICATIONS

This application claims the benefit of U.S. Provisional Application Ser. No. 60/516,338 filed Oct. 31, 2003.

The present disclosure is generally directed to antiviral compounds, and more specifically directed to compounds which can inhibit the function of HCV polymerase encoded by Hepatitis C virus (HCV), compositions comprising such compounds and methods for inhibiting the function of HCV polymerase.

HCV is a major human pathogen, infecting an estimated 170 million persons worldwide—roughly five times the number infected by human immunodeficiency virus type 1. A substantial fraction of these HCV infected individuals develop serious progressive liver disease, including cirrhosis and hepatocellular carcinoma (Lauer, G. M.; Walker, B. D. N. Engl. J. Med. 2001, 345, 41-52).

Presently, the most effective HCV therapy employs a combination of alpha-interferon and ribavirin, leading to sustained efficacy in 40% of patients. (Poynard, T. et al. Lancet 1998, 352, 1426-1432). Recent clinical results demonstrate that pegylated alpha-interferon is superior to unmodified alpha-interferon as monotherapy (Zeuzem, S. et al. N. Engl J. Med. 2000, 343, 1666-1672). However, even with experimental therapeutic regimens involving combinations of pegylated alpha-interferon and ribavirin, a substantial fraction of patients do not have a sustained reduction in viral load. Thus, there is a clear and long-felt need to develop effective therapeutics for treatment of HCV infection.

HCV is a positive-stranded RNA virus. Based on a comparison of the deduced amino acid sequence and the extensive similarity in the 5′ untranslated region, HCV has been classified as a separate genus in the Flaviviridae family. All members of the Flaviviridae family have enveloped virions that contain a positive stranded RNA genome encoding all known virus-specific proteins via translation of a single, uninterrupted, open reading frame.

Considerable heterogeneity is found within the nucleotide and encoded amino acid sequence throughout the HCV genome. At least six major genotypes have been characterized, and more than 50 subtypes have been described. The major genotypes of HCV differ in their distribution worldwide, and the clinical significance of the genetic heterogeneity of HCV remains elusive despite numerous studies of the possible effect of genotypes on pathogenesis and therapy.

The single strand HCV RNA genome is approximately 9500 nucleotides in length and has a single open reading frame (ORF) encoding a single large polyprotein of about 3000 amino acids. In infected cells, this polyprotein is cleaved at multiple sites by cellular and viral proteases to produce the structural and non-structural (NS) proteins. In the case of HCV, the generation of mature non-structural proteins (NS2, NS3, NS4A, NS4B, NS5A, and NS5B) is effected by two viral proteases. The first one is believed to be a metalloprotease and cleaves at the NS2-NS3 junction; the second one is a serine protease contained within the N-terminal region of NS3 (also referred to herein as NS3 protease) and mediates all the subsequent cleavages downstream of NS3, both in cis, at the NS3-NS4A cleavage site, and in trans, for the remaining NS4A-NS4B, NS4B-NS5A, NS5A-NS5B sites. The NS4A protein appears to serve multiple functions, acting as a cofactor for the NS3 protease and possibly assisting in the membrane localization of NS3 and other viral replicase components. The complex formation of the NS3 protein with NS4A seems necessary to the processing events, enhancing the proteolytic efficiency at all of the sites. The NS3 protein also exhibits nucleoside triphosphatase and RNA helicase activities. NS5B (also referred to herein as HCV polymerase) is a RNA-dependent RNA polymerase that is involved in the replication of HCV.

Among the compounds that have demonstrated efficacy in inhibiting HCV replication, as selective HCV serine protease inhibitors, are the peptide compounds disclosed in U.S. Pat. Ser. No. 6,323,180. NS5B polymerase inhibitors have also demonstrated activity. However, none of these compounds have, to date, progressed beyond clinical trials (De Clercq, E. J. Clin. Virol. 2001, 22, 73-89).

Compounds useful for treating HCV-infected patients are desired which selectively inhibit HCV viral replication. In particular, compounds which are effective to inhibit the function of the NS5B protein are desired. The HCV NS5B protein is described, for example, in “Structural Analysis of the Hepatitis C Virus RNA Polymerase in Complex with Ribonucleotides:, S. Bressanelli, et al., Journal of Virology, 2002, 3482-3492 and Defrancesco & Rice, Clinics in Liver Disease, 2003, 7, 211-242.

In one embodiment the present disclosure provides a compound of formula (I) 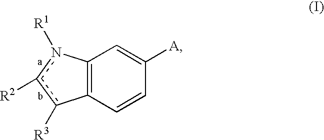
or a pharmaceutically acceptable salt thereof, wherein 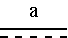
is a single or double bond; 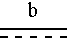
is a single or double bond;
provided that at least one of 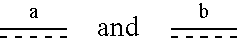
   is a single bond;

A is selected from —NH₂, (NR^{a}R^{b})sulfonyl, an unsaturated 5-membered ring having 3 or 4 heteroatoms selected from nitrogen, oxygen, and sulfur, wherein the ring is optionally substituted with one or two substituents selected from oxo and (thio)oxo, 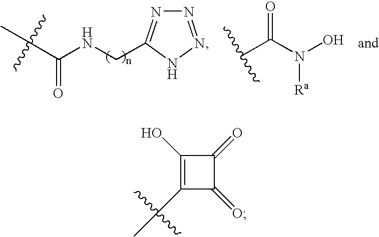
wherein n is 0 to 3 and 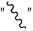
denotes the point of attachment of the substituent to the parent molecule;
R¹ is selected from hydrogen, alkenyl, alkyl, and alkynyl;
R² is selected from aryl and heteroaryl;
R³ is selected from cycloalkenyl and cycloalkyl;
R^{a} is selected from hydrogen, alkenyl, alkyl, and alkynyl; and
R^{b} is selected from hydroxy and alkylcarbonyl.

In another embodiment the present disclosure provides a compound of formula (I) wherein A is selected from —NH₂, (NR^{a}R^{b})sulfonyl,

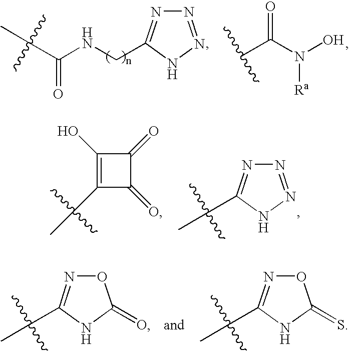

In another embodiment the present disclosure provides a compound of formula (I) wherein A is selected from

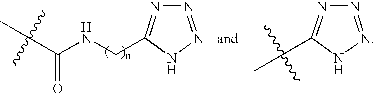

In another embodiment the present disclosure provides a compound of formula (I) wherein A is

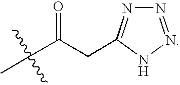

In another embodiment the present disclosure provides a compound of formula (I) wherein A is

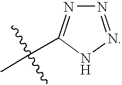

In another embodiment the present disclosure provides a compound of formula (II) 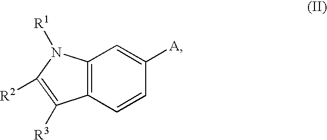
or a pharmaceutically acceptable salt thereof, wherein
A is selected from —NH₂, (NR^{a}R^{b})sulfonyl, 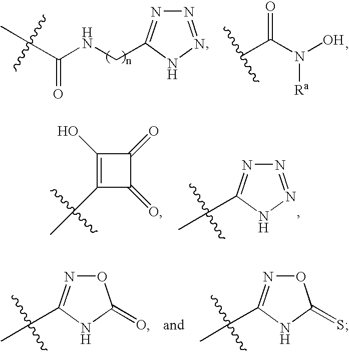
   wherein n is 0 to 3 and “ [symbol] ” denotes the point of attachment of the substituent to the parent molecule;
R¹ is selected from hydrogen and alkyl;
R² is heteroaryl;
R³ is cycloalkyl;
R^{a} is selected from hydrogen, alkenyl, alkyl, and alkynyl; and
R^{b} is selected from hydroxy and alkylcarbonyl.

In another embodiment the present disclosure provides a compound of formula (III) 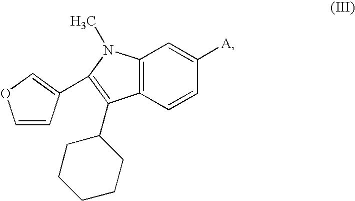
or a pharmaceutically acceptable salt thereof, wherein
A is selected from

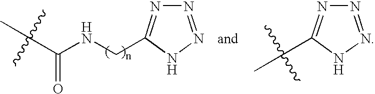

In another embodiment the present disclosure provides a composition comprising a compound of formula (I), or a pharmaceutically acceptable salt thereof, and a pharmaceutically acceptable carrier. In another embodiment the composition further comprises an interferon and ribavirin.

In another embodiment the present disclosure provides a composition comprising a compound of formula (I), or a pharmaceutically acceptable salt thereof, a pharmaceutically acceptable carrier, and another compound having anti-HCV activity. In another embodiment the other compound having anti-HCV activity is an interferon. In another embodiment the interferon is selected from interferon alpha 2B, pegylated interferon alpha, consensus interferon, interferon alpha 2A, and lymphoblastiod interferon tau.

In another embodiment the present disclosure provides a composition comprising a compound of formula (I), or a pharmaceutically acceptable salt thereof, a pharmaceutically acceptable carrier, and another compound having anti-HCV activity wherein the other compound having anti-HCV activity is selected from interleukin 2, interleukin 6, interleukin 12, a compound that enhances the development of a type 1 helper T cell response, interfering RNA, anti-sense RNA, Imiqimod, ribavirin, an inosine 5′-monophospate dehydrogenase inhibitor, amantadine, and rimantadine.

In another embodiment the present disclosure provides a composition comprising a compound of formula (I), or a pharmaceutically acceptable salt thereof, a pharmaceutically acceptable carrier, and another compound having anti-HCV activity wherein the other compound having anti-HCV activity is a small molecule compound.

In another embodiment the present disclosure provides a composition comprising a compound of formula (I), or a pharmaceutically acceptable salt thereof, a pharmaceutically acceptable carrier, and another compound having anti-HCV activity wherein the other compound having anti-HCV activity is effective to inhibit the function of a target selected from HCV metalloprotease, HCV serine protease, HCV polymerase, HCV helicase, HCV NS4B protein, HCV entry, HCV assembly, HCV egress, HCV NS5A protein, IMPDH and a nucleoside analog for the treatment of an HCV infection.

In another embodiment the present disclosure provides a method of inhibiting the function of the HCV NS5B protein comprising contacting the HCV NS5B protein with a compound of formula (I) or a pharmaceutically acceptable salt thereof.

In another embodiment the present disclosure provides a method of treating an HCV infection in a patient, comprising administering to the patient a therapeutically effective amount of the compound of formula (I) or a pharmaceutically acceptable salt thereof. In another embodiment the compound is effective to inhibit the function of the HCV NS5B protein.

In another embodiment the present disclosure provides a method of treating an HCV infection in a patient, comprising administering to the patient a therapeutically effective amount of the compound of formula (I), or a pharmaceutically acceptable salt thereof, wherein the method further comprises administering another compound having anti-HCV activity prior to, after, or simultaneously with the compound of formula (I). In another embodiment the other compound having anti-HCV activity is an interferon. In another embodiment the interferon is selected from interferon alpha 2B, pegylated interferon alpha, consensus interferon, interferon alpha 2A, and lymphoblastiod interferon tau. In another embodiment the other compound having anti-HCV activity is selected from interleukin 2, interleukin 6, interleukin 12, a compound that enhances the development of a type 1 helper T cell response, interfering RNA, anti-sense RNA, Imiqimod, ribavirin, an inosine 5′-monophospate dehydrogenase inhibitor, amantadine, and rimantadine.

In another embodiment the present disclosure provides a method of treating an HCV infection in a patient, comprising administering to the patient a therapeutically effective amount of the compound of formula (I), or a pharmaceutically acceptable salt thereof, wherein the method further comprises administering another compound having anti-HCV activity prior to, after, or simultaneously with the compound of formula (I), wherein the other compound having anti-HCV activity is a small molecule. In another embodiment the compound having anti-HCV activity is effective to inhibit the function of a target selected from HCV metalloprotease, HCV serine protease, HCV polymerase, HCV helicase, HCV NS4B protein, HCV entry, HCV assembly, HCV egress, HCV NS5A protein, IMPDH and a nucleoside analog for the treatment of an HCV infection.

In another embodiment the present disclosure provides a method of treating an HCV infection in a patient, comprising administering to the patient a therapeutically effective amount of the compound of formula (I), or a pharmaceutically acceptable salt thereof, wherein the method further comprises administering another compound having anti-HCV activity prior to, after, or simultaneously with the compound of formula (I), wherein the other compound having anti-HCV activity is effective to inhibit the function of a target in the HCV life cycle other than the HCV NS5B protein.

In another embodiment the present disclosure provides the use of a compound of formula (I) for the manufacture of a medicament for treating HCV infection in a patient.

In another embodiment the present disclosure provides the use of a composition comprising a compound of formula (I), or a pharmaceutically acceptable salt thereof, and a pharmaceutically acceptable carrier for the manufacture of a medicament for treating HCV infection in a patient.

As used in the present specification, the following terms have the meanings indicated:

The term “alkenyl,” as used herein, refers to a straight or branched chain group of two to six carbon atoms containing at least one carbon-carbon double bond.

The term “alkoxy,” as used herein, refers to an alkyl group attached to the parent molecular moiety through an oxygen atom.

The term “alkyl,” as used herein, refers to a group derived from a straight or branched chain saturated hydrocarbon containing from one to six carbon atoms.

The term “alkylcarbonyl,” as used herein, refers to an alkyl group attached to the parent molecular moiety through a carbonyl group.

The term “alkynyl,” as used herein, refers to a straight or branched chain hydrocarbon of two to six carbon atoms containing at least one carbon-carbon triple bond.

The term “aryl,” as used herein, refers to a phenyl group, or a bicyclic fused ring system wherein one or both of the rings is a phenyl group. Bicyclic fused ring systems consist of a phenyl group fused to a four- to six-membered aromatic or non-aromatic carbocyclic ring. The aryl groups of the present disclosure can be attached to the parent molecular moiety through any substitutable carbon atom in the group. Representative examples of aryl groups include, but are not limited to, indanyl, indenyl, naphthyl, phenyl, and tetrahydronaphthyl. The aryl groups of the present disclosure can be optionally substituted with one or two substituents independently selected from alkoxy, alkyl, halo, haloalkoxy, and haloalkyl.

The term “carbonyl,” as used herein, refers to —C(O).

The terms “halo” and “halogen,” as used herein, refer to F, Cl, Br, or I.

The term “haloalkoxy,” as used herein, refers to a haloalkyl group attached to the parent molecular moiety through an oxygen atom.

The term “haloalkyl,” as used herein, refers to an alkyl group substituted by one, two, three, or four halogen atoms.

The term “heteroaryl,” as used herein, refers to an aromatic five- or six-membered ring where at least one atom is selected from N, O, and S, and the remaining atoms are carbon. The term “heteroaryl” also includes bicyclic systems where a heteroaryl ring is fused to a four- to six-membered aromatic or non-aromatic ring containing zero, one, or two additional heteroatoms selected from N, O, and S. The heteroaryl groups are attached to the parent molecular moiety through any substitutable carbon or nitrogen atom in the group. Representative examples of heteroaryl groups include, but are not limited to, benzoxadiazolyl, benzoxazolyl, benzofuranyl, benzothienyl, furanyl, imidazolyl, indazolyl, indolyl, isoxazolyl, isoquinolinyl, isothiazolyl, naphthyridinyl, oxadiazolyl, oxazolyl, pyridinyl, pyridazinyl, pyrimidinyl, pyrazinyl, pyrazolyl, pyrrolyl, quinolinyl, thiazolyl, thienopyridinyl, thienyl, triazolyl, thiadiazolyl, and triazinyl. The heteroaryl groups of the present disclosure can be optionally substituted with one or two substituents independently selected from alkoxy, alkyl, halo, haloalkoxy, and haloalkyl.

The term “hydroxy,” as used herein, refers to —OH.

The term “—NR^{a}R^{b}, as used herein, refers to two groups, R^{a} and R^{b}, which are attached to the parent molecular moiety through a nitrogen atom. R^{a} is selected from hydrogen, alkenyl, alkyl, and alkynyl; and R^{b} is selected from hydroxy and alkylcarbonyl.

The term “(NR^{a}R^{b})sulfonyl,” as used herein, refers to an —NR^{a}R^{b} group attached to the parent molecular moiety through a sulfonyl group.

The term “oxo,” as used herein, refers to ═O.

The term “(thio)oxo,” as used herein, refers to ═S.

The term “sulfonyl,” as used herein, refers to —SO₂—.

The compounds of the present invention can exist as pharmaceutically acceptable salts. The term “pharmaceutically acceptable salt,” as used herein, represents salts or zwitterionic forms of the compounds of the present invention which are water or oil-soluble or dispersible, which are, within the scope of sound medical judgment, suitable for use in contact with the tissues of patients without excessive toxicity, irritation, allergic response, or other problem or complication commensurate with a reasonable benefit/risk ratio, and are effective for their intended use The salts can be prepared during the final isolation and purification of the compounds or separately by reacting a suitable nitrogen atom with a suitable acid. Representative acid addition salts include acetate, adipate, alginate, citrate, aspartate, benzoate, benzenesulfonate, bisulfate, butyrate, camphorate, camphorsulfonate; digluconate, glycerophosphate, hemisulfate, heptanoate, hexanoate, formate, fumarate, hydrochloride, hydrobromide, hydroiodide, 2-hydroxyethanesulfonate, lactate, maleate, mesitylenesulfonate, methanesulfonate, naphthylenesulfonate, nicotinate, 2-naphthalenesulfonate, oxalate, palmoate, pectinate, persulfate, 3-phenylproprionate, picrate, pivalate, propionate, succinate, tartrate, trichloroacetate, trifluoroacetate, phosphate, glutamate, bicarbonate, para-toluenesulfonate, and undecanoate. Examples of acids which can be employed to form pharmaceutically acceptable addition salts include inorganic acids such as hydrochloric, hydrobromic, sulfuric, and phosphoric, and organic acids such as oxalic, maleic, succinic, and citric.

When it is possible that, for use in therapy, therapeutically effective amounts of a compound of formula (I), as well as pharmaceutically acceptable salts thereof, may be administered as the raw chemical, it is possible to present the active ingredient as a pharmaceutical composition. Accordingly, the invention further provides pharmaceutical compositions, which include therapeutically effective amounts of compounds of formula (I) or pharmaceutically acceptable salts thereof, and one or more pharmaceutically acceptable carriers, diluents, or excipients. The term “therapeutically effective amount,” as used herein, refers to the total amount of each active component that is sufficient to show a meaningful patient benefit, e.g., a sustained reduction in viral load. When applied to an individual active ingredient, administered alone, the term refers to that ingredient alone. When applied to a combination, the term refers to combined amounts of the active ingredients that result in the therapeutic effect, whether administered in combination, serially, or simultaneously. The compounds of formula (I) and pharmaceutically acceptable salts thereof, are as described above. The carrier(s), diluent(s), or excipient(s) must be acceptable in the sense of being compatible with the other ingredients of the formulation and not deleterious to the recipient thereof. In accordance with another aspect of the invention there is also provided a process for the preparation of a pharmaceutical formulation including admixing a compound of formula (I), or a pharmaceutically acceptable salt thereof, with one or more pharmaceutically acceptable carriers, diluents, or excipients. The term “pharmaceutically acceptable,” as used herein, refers to those compounds, materials, compositions, and/or dosage forms which are, within the scope of sound medical judgment, suitable for use in contact with the tissues of patients without excessive toxicity, irritation, allergic response, or other problem or complication commensurate with a reasonable benefit/risk ratio, and are effective for their intended use.

The term “patient” includes both human and other mammals.

The term “treating” refers to: (i) preventing a disease, disorder or condition from occurring in a patient which may be predisposed to the disease, disorder and/or condition but has not yet been diagnosed as having it; (ii) inhibiting the disease, disorder or condition, i.e., arresting its development; and (iii) relieving the disease, disorder or condition, i.e., causing regression of the disease, disorder and/or condition.

Certain compounds of the present disclosure, and their salts, may also exist in the form of solvates with water, for example hydrates, or with organic solvents such as methanol, ethanol or acetonitrile to form, respectively, a methanolate, ethanolate or acetonitrilate. The present disclosure includes each solvate and mixtures thereof.

Certain compounds of the present disclosure may also exist in different stable conformational forms which may be separable. Torsional asymmetry due to restricted rotation about an asymmetric single bond, for example because of steric hindrance or ring strain, may permit separation of different conformers. The present disclosure includes each conformational isomer of these compounds and mixtures thereof.

Pharmaceutical formulations may be presented in unit dose forms containing a predetermined amount of active ingredient per unit dose. Dosage levels of between about 0.01 and about 250 milligram per kilogram (“mg/kg”) body weight per day, preferably between about 0.05 and about 100 mg/kg body weight per day of the compounds of the invention are typical in a monotherapy for the prevention and treatment of HCV mediated disease. Typically, the pharmaceutical compositions of this invention will be administered from about 1 to about 5 times per day or alternatively, as a continuous infusion. Such administration can be used as a chronic or acute therapy. The amount of active ingredient that may be combined with the carrier materials to produce a single dosage form will vary depending on the condition being treated, the severity of the condition, the time of administration, the route of administration, the rate of excretion of the compound employed, the duration of treatment, and the age, gender, weight, and condition of the patient. Preferred unit dosage formulations are those containing a daily dose or sub-dose, as herein above recited, or an appropriate fraction thereof, of an active ingredient. Generally, treatment is initiated with small dosages substantially less than the optimum dose of the compound. Thereafter, the dosage is increased by small increments until the optimum effect under the circumstances is reached. In general, the compound is most desirably administered at a concentration level that will generally afford antivirally effective results without causing any harmful or deleterious side effects.

When the compositions of this invention comprise a combination of a compound of the invention and one or more additional therapeutic or prophylactic agent, both the compound and the additional agent are usually present at dosage levels of between about 10 to 150%, and more preferably between about 10 and 80% of the dosage normally administered in a monotherapy regimen.

Pharmaceutical formulations may be adapted for administration by any appropriate route, for example by the oral (including buccal or sublingual), rectal, nasal, topical (including buccal, sublingual, or transdermal), vaginal, or parenteral (including subcutaneous, intracutaneous, intramuscular, intra-articular, intrasynovial, intrasternal, intrathecal, intralesional, intravenous, or intradermal injections or infusions) route. Such formulations may be prepared by any method known in the art of pharmacy, for example by bringing into association the active ingredient with the carrier(s) or excipient(s). Oral administration or administration by injection are preferred.

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil emulsions.

For instance, for oral administration in the form of a tablet or capsule, the active drug component can be combined with an oral, non-toxic pharmaceutically acceptable inert carrier such as ethanol, glycerol, water, and the like. Powders are prepared by comminuting the compound to a suitable fine size and mixing with a similarly comminuted pharmaceutical carrier such as an edible carbohydrate, as, for example, starch or mannitol. Flavoring, preservative, dispersing, and coloring agent can also be present.

Capsules are made by preparing a powder mixture, as described above, and filling formed gelatin sheaths. Glidants and lubricants such as colloidal silica, talc, magnesium stearate, calcium stearate, or solid polyethylene glycol can be added to the powder mixture before the filling operation. A disintegrating or solubilizing agent such as agar-agar, calcium carbonate, or sodium carbonate can also be added to improve the availability of the medicament when the capsule is ingested.

Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, and the like. Lubricants used in these dosage forms include sodium oleate, sodium chloride, and the like. Disintegrators include, without limitation, starch, methyl cellulose, agar, betonite, xanthan gum, and the like. Tablets are formulated, for example, by preparing a powder mixture, granulating or slugging, adding a lubricant and disintegrant, and pressing into tablets. A powder mixture is prepared by mixing the compound, suitable comminuted, with a diluent or base as described above, and optionally, with a binder such as carboxymethylcellulose, an aliginate, gelating, or polyvinyl pyrrolidone, a solution retardant such as paraffin, a resorption accelerator such as a quaternary salt and/or and absorption agent such as betonite, kaolin, or dicalcium phosphate. The powder mixture can be granulated by wetting with a binder such as syrup, starch paste, acadia mucilage, or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc, or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material, and a polish coating of wax can be provided. Dyestuffs can be added to these coatings to distinguish different unit dosages.

Oral fluids such as solution, syrups, and elixirs can be prepared in dosage unit form so that a given quantity contains a predetermined amount of the compound. Syrups can be prepared by dissolving the compound in a suitably flavored aqueous solution, while elixirs are prepared through the use of a non-toxic vehicle. Solubilizers and emulsifiers such as ethoxylated isostearyl alcohols and polyoxyethylene sorbitol ethers, preservatives, flavor additive such as peppermint oil or natural sweeteners, or saccharin or other artificial sweeteners, and the like can also be added.

Where appropriate, dosage unit formulations for oral administration can be microencapsulated. The formulation can also be prepared to prolong or sustain the release as for example by coating or embedding particulate material in polymers, wax, or the like.

The compounds of formula (I), and pharmaceutically acceptable salts thereof, can also be administered in the form of liposome delivery systems, such as small unilamellar vesicles, large unilamellar vesicles, and multilamellar vesicles. Liposomes can be formed from a variety of phopholipids, such as cholesterol, stearylamine, or phophatidylcholines.

The compounds of formula (I) and pharmaceutically acceptable salts thereof may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamidephenol, polyhydroxyethylaspartamidephenol, or polyethyleneoxidepolylysine substituted with palitoyl residues. Furthermore, the compounds may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates, and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986).

Pharmaceutical formulations adapted for topical administration may be formulated as ointments, creams, suspensions, lotions, powders, solutions, pastes, gels, sprays, aerosols, or oils.

For treatments of the eye or other external tissues, for example mouth and skin, the formulations are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles, and mouth washes.

Pharmaceutical formulations adapted for rectal administration may be presented as suppositories or as enemas.

Pharmaceutical formulations adapted for nasal administration wherein the carrier is a solid include a course powder having a particle size for example in the range 20 to 500 microns which is administered in the manner in which snuff is taken, i.e., by rapid inhalation through the nasal passage from a container of the powder held close up to the nose. Suitable formulations wherein the carrier is a liquid, for administration as a nasal spray or nasal drops, include aqueous or oil solutions of the active ingredient.

Pharmaceutical formulations adapted for administration by inhalation include fine particle dusts or mists, which may be generated by means of various types of metered, dose pressurized aerosols, nebulizers, or insufflators.

Pharmaceutical formulations adapted for vaginal administration may be presented as pessaries, tampons, creams, gels, pastes, foams, or spray formulations.

Pharmaceutical formulations adapted for parenteral administration include aqueous and non-aqueous sterile injection solutions which may contain anti-oxidants, buffers, bacteriostats, and soutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets.

It should be understood that in addition to the ingredients particularly mentioned above, the formulations may include other agents conventional in the art having regard to the type of formulation in question, for example those suitable for oral administration may include flavoring agents.

As used herein, the term “anti-HCV activity” means the compound is effective to inhibit the function of a target selected from HCV metalloprotease, HCV serine protease, HCV polymerase, HCV helicase, HCV NS4B protein, HCV entry, HCV assembly, HCV egress, HCV NS5A protein, IMPDH and a nucleoside analog for the treatment of an HCV infection. Often, the other compound having anti-HCV activity is effective to inhibit the function of target in the HCV life cycle other than the HCV NS5B protein.

Certain illustrative HCV inhibitor compounds which can be administered with the compounds of the present disclosure include those disclosed in the following publications: WO 02/04425 A2 published Jan. 17, 2002, WO 03/007945 A1 published Jan. 30, 2003, WO 03/010141 A2 published Feb. 6, 2003, WO 03/010142 A2 published Feb. 6, 2003, WO 03/010143 A1 published Feb. 6, 2003, WO 03/000254 A1 published Jan. 3, 2003, WO 01/32153 A2 published May 10, 2001, WO 00/06529 published Feb. 10, 2000, WO 00/18231 published Apr. 6, 2000, WO 00/10573 published Mar. 2, 2000, WO 00/13708 published Mar. 16, 2000, WO 01/85172 A1 published Nov. 15, 2001, WO 03/037893 A1 published May 8, 2003, WO 03/037894 A1 published May 8, 2003, WO 03/037895 A1 published May 8, 2003, WO 02/100851 A2 published Dec. 19, 2002, WO 02/100846 A1 published Dec. 19, 2002, EP 1256628 A2 published Nov. 13, 2002, WO 99/01582 published Jan. 14, 1999, WO 00/09543 published Feb. 24, 2000.

Table 1 below lists some illustrative examples of compounds that can be administered with the compounds of this disclosure. The compounds of the disclosure can be administered with other anti-HCV activity compounds in combination therapy, either jointly or separately, or by combining the compounds into a composition.

TABLE 1

| | Type of Inhibitor or | |
|---|---|---|
| Brand Name | Target | Source Company |
| Omega IFN | IFN-ω | BioMedicines Inc., |
| | | Emeryville, CA |
| BILN-2061 | serine protease | Boehringer Ingelheim |
| | inhibitor | Pharma KG, Ingelheim, |
| | | Germany |
| Summetrel | antiviral | Endo Pharmaceuticals |
| | | Holdings Inc., Chadds |
| | | Ford, PA |
| Roferon A | IFN-α2a | F. Hoffmann-La Roche |
| | | LTD, Basel, Switzerland |
| Pegasys | PEGylated IFN-α2a | F. Hoffmann-La Roche |
| | | LTD, Basel, Switzerland |
| Pegasys and Ribavirin | PEGylated IFN- | F. Hoffmann-La Roche |
| | α2a/ribavirin | LTD, Basel, Switzerland |
| CellCept | HCV IgG | F. Hoffmann-La Roche |
| | immunosuppressant | LTD, Basel, Switzerland |
| Wellferon | lymphoblastoid IFN- | GlaxoSmithKline plc, |
| | αn1 | Uxbridge, UK |
| Albuferon - α | albumin IFN-α2b | Human Genome |
| | | Sciences Inc., Rockville, |
| | | MD |
| Levovirin | ribavirin | ICN Pharmaceuticals, |
| | | Costa Mesa, CA |
| IDN-6556 | caspase inhibitor | Idun Pharmaceuticals |
| | | Inc., San Diego, CA |
| IP-501 | antifibrotic | Indevus Pharmaceuticals |
| | | Inc., Lexington, MA |
| Actimmune | INF-γ | InterMune Inc., |
| | | Brisbane, CA |
| Infergen A | IFN alfacon-1 | InterMune |
| | | Pharmaceuticals Inc., |
| | | Brisbane, CA |
| ISIS 14803 | antisense | ISIS Pharmaceuticals |
| | | Inc, Carlsbad, CA/Elan |
| | | Phamaceuticals Inc., |
| | | New York, NY |
| JTK-003 | RdRp inhibitor | Japan Tobacco Inc., |
| | | Tokyo, Japan |
| Pegasys and Ceplene | PEGylated IFN-α2a/ | Maxim Pharmaceuticals |
| | immune modulator | Inc., San Diego, CA |
| Ceplene | immune modulator | Maxim Pharmaceuticals |
| | | Inc., San Diego, CA |
| Civacir | HCV IgG | Nabi |
| | immunosuppressant | Biopharmaceuticals Inc., |
| | | Boca Raton, FL |
| Intron A and Zadaxin | IFN-α2b/α1-thymosin | RegeneRx |
| | | Biopharmiceuticals Inc., |
| | | Bethesda, MD/ |
| | | SciClone |
| | | Pharmaceuticals Inc, |
| | | San Mateo, CA |
| Levovirin | IMPDH inhibitor | Ribapharm Inc., Costa |
| | | Mesa, CA |
| Viramidine | IMPDH inhibitor | Ribapharm Inc., Costa |
| | | Mesa, CA |
| Heptazyme | ribozyme | Ribozyme |
| | | Pharmaceuticals Inc., |
| | | Boulder, CO |
| Intron A | IFN-α2b | Schering-Plough |
| | | Corporation, |
| | | Kenilworth, NJ |
| PEG-Intron | PEGylated IFN-α2b | Schering-Plough |
| | | Corporation, |
| | | Kenilworth, NJ |
| Rebetron | IFN-α2b/ribavirin | Schering-Plough |
| | | Corporation, |
| | | Kenilworth, NJ |
| Ribavirin | ribavirin | Schering-Plough |
| | | Corporation, |
| | | Kenilworth, NJ |
| PEG-Intron/Ribavirin | PEGylated IFN- | Schering-Plough |
| | α2b/ribavirin | Corporation, |
| | | Kenilworth, NJ |
| Zadazim | immune modulator | SciClone |
| | | Pharmaceuticals Inc., |
| | | San Mateo, CA |
| Rebif | IFN-β1a | Serono, Geneva, |
| | | Switzerland |
| IFN-β and EMZ701 | IFN-β and EMZ701 | Transition Therapeutics |
| | | Inc., Ontario, Canada |
| T67 | β-tubulin inhibitor | Tularik Inc., South San |
| | | Francisco, CA |
| VX-497 | IMPDH inhibitor | Vertex Pharmaceuticals |
| | | Inc., Cambridge, MA |
| VX-950/LY-570310 | serine protease | Vertex Pharmaceuticals |
| | inhibitor | Inc., Cambridge, MA/ |
| | | Eli Lilly and Co. Inc., |
| | | Indianapolis, IN |
| Omniferon | natural IFN-α | Viragen Inc., Plantation, |
| | | FL |
| XTL-002 | monoclonal antibody | XTL |
| | | Biopharmaceuticals |
| | | Ltd., Rehovot, Isreal |

The compounds of the disclosure may also be used as laboratory reagents. Compounds may be instrumental in providing research tools for designing of viral replication assays, validation of animal assay systems and structural biology studies to further enhance knowledge of the HCV disease mechanisms. Further, the compounds of the present disclosure are useful in establishing or determining the binding site of other antiviral compounds, for example, by competitive inhibition.

The compounds of this disclosure may also be used to treat or prevent viral contamination of materials and therefore reduce the risk of viral infection of laboratory or medical personnel or patients who come in contact with such materials, e.g., blood, tissue, surgical instruments and garments, laboratory instruments and garments, and blood collection or transfusion apparatuses and materials.

The compounds and processes of the present invention will be better understood in connection with the following synthetic schemes which illustrate the methods by which the compounds of the invention may be prepared. Starting materials can be obtained from commercial sources or prepared by well-established literature methods known to those of ordinary skill in the art. It will be readily apparent to one of ordinary skill in the art that the compounds defined above can be synthesized by substitution of the appropriate reactants and agents in the syntheses shown below. It will also be readily apparent to one skilled in the art that the selective protection and deprotection steps, as well as the order of the steps themselves, can be carried out in varying order, depending on the nature of R¹, R², R³, R^{a}, R^{b}, n, and A to successfully complete the syntheses below. The groups R¹, R², R³, R^{a}, R^{b}, n, and A are as defined above unless otherwise noted below.

Abbreviations used in the following schemes and examples are: DMF for N,N-dimethylformamide; DMSO for dimethylsulfoxide; DPPA for diphenylphosphoryl azide; and Sn(n-Bu)₃ for tri-(n-butyl)stannyl; TBTU for 2-(1H-benzotriazol-1-yl)-1,1,3,3-tetramethyluronium tetrafluoroborate; TEA for triethylamine; TFA for trifluoroacetic acid; and THF for tetrahydrofuran.

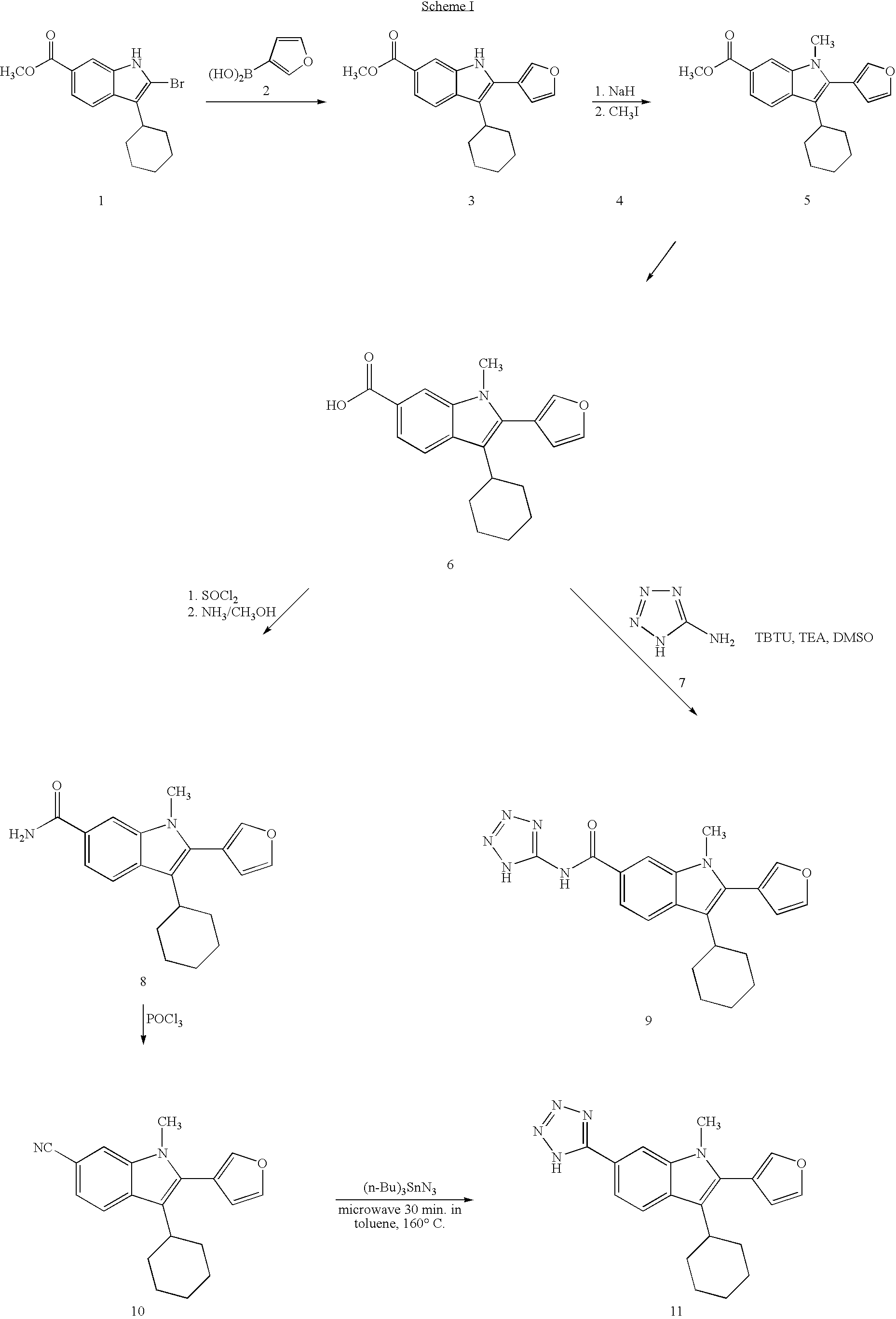

Scheme I depicts the syntheses of compounds of formulae 9 and 11. The syntheses of intermediates 1, 3, 5, and 6 are described, for example, in WO 03/010141. Other compounds of the present disclosure can be synthesized from analogous intermediates. For example, 3-furylboronic acid (2) can be replaced by other heterocyclic and arylboronic acids to afford the corresponding R² substituents of the present disclosure. Similarly, replacement of iodomethane (4) with other alkylating species will afford the corresponding R¹ substituents of the present disclosure.

Treatment of a mixture of carboxylic acid 6 in methylene chloride with thionyl chloride and a catalytic amount of N,N-dimethylformamide provides the acid chloride which upon treatment with methanolic ammonia affords amide 8 which is dehydrated with phosphorous oxychloride to afford nitrile 10. A solution of 10 in toluene is microwaved with azidotributyltin at 160° C. to afford tetrazole 11. The tranformations of carboxylic acids into tetrazoles which proceed through nitrites are well documented in the literature and numerous alternative reagents are known to practitioners of the art. For example, see: “Recent Developments in Tetrazole Chemistry. A Review”, S. J. Wittenberger, Organic Preparations and Procedures Int., 26 (5), 499-531 (1994).

Carboxylic acid 6, after activation with 2-(1H-benzotriazol-1-yl)-1,1,3,3-tetramethyluronium tetrafluoroborate (TBTU), is coupled with 5-aminotetrazole (7) to afford 9. Alternatively, carboxylic acid 6 can be activated by conversion to the acid chloride, mixed anhydride, or by other reagents used for the synthesis of peptides such as carbonyldiimidazole, dicyclohexylcarbodiimide and the like.

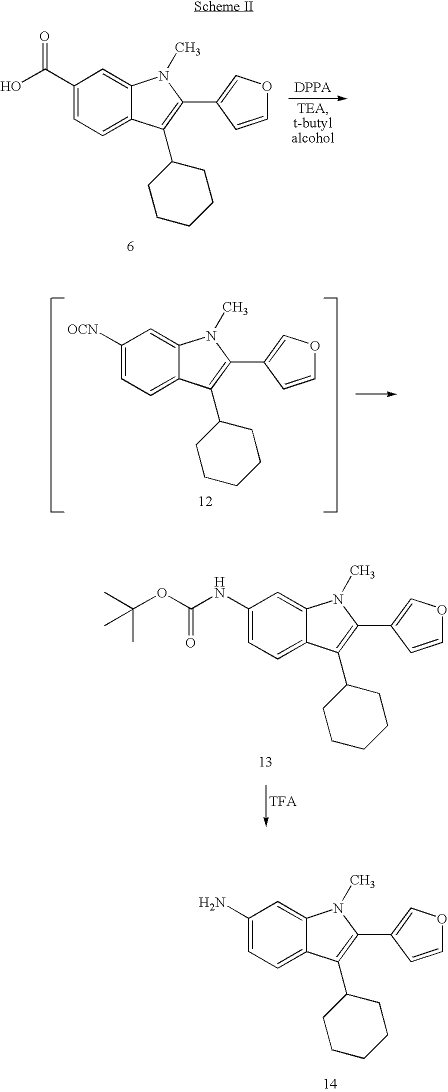

Compounds of formula I wherein A is NH₂ are synthesized as shown in Scheme II.

Heating a mixture of carboxylic acid 6 in t-butyl alcohol and triethylamine with diphenylphosphoryl azide at about 140° C. gives carbamate 13 which upon treatment with trifluoroacetic acid generates indole 14 as the TFA salt.

Those skilled in the art will recognize numerous variations of this procedure. In the example shown the intermediate isocyanate 12 is trapped as the carbamate 13 which facilitates purification. Utilization of benzyl alcohol will result in isolation of a benzyl carbamate which serves as a protecting group removed by hydrogenolysis to afford 14.

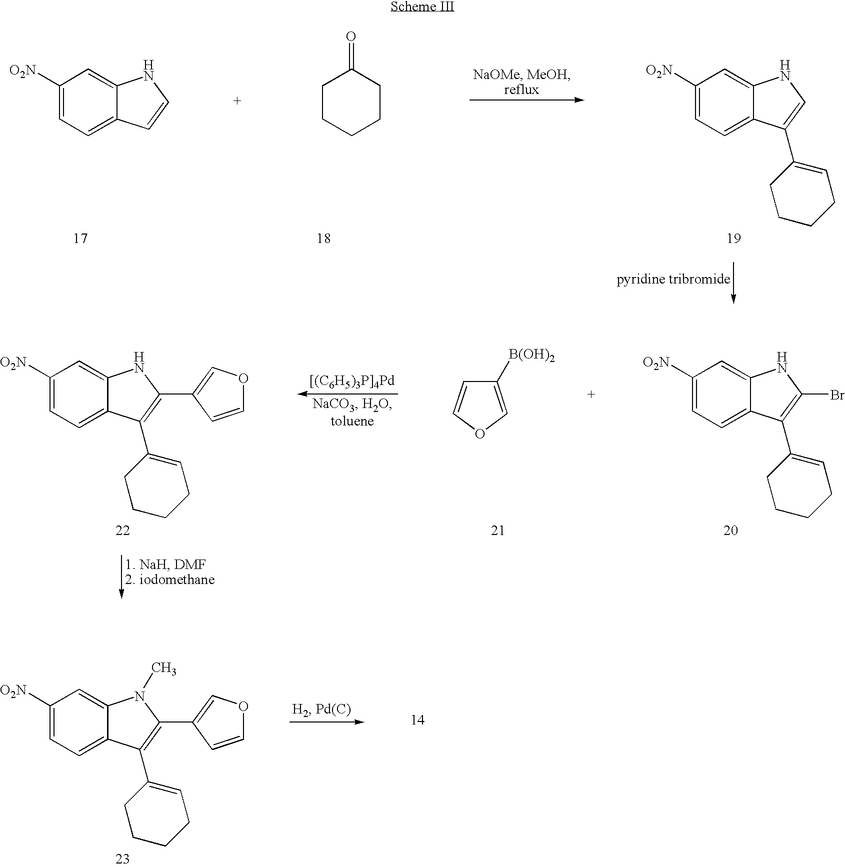

Scheme III depicts amine 14 being synthesized starting from 6-nitroindole 17 where condensation of 6-nitroindole 17 with cyclohexanone (18) in the presence of methanolic sodium methoxide affords cyclohexenyl derivative 19 which can be brominated with pyridine tribromide at 0° C. in THF to afford 20. The bromo derivative 20 and 3-furylboronic acid (21) are cross coupled using the Suzuki Reaction to afford 22 which is alkylated with iodomethane to give 23. Reduction of both the nitro group and cyclohexene bond with hydrogen and palladium in an inert solvent gives the aminoindole 14.

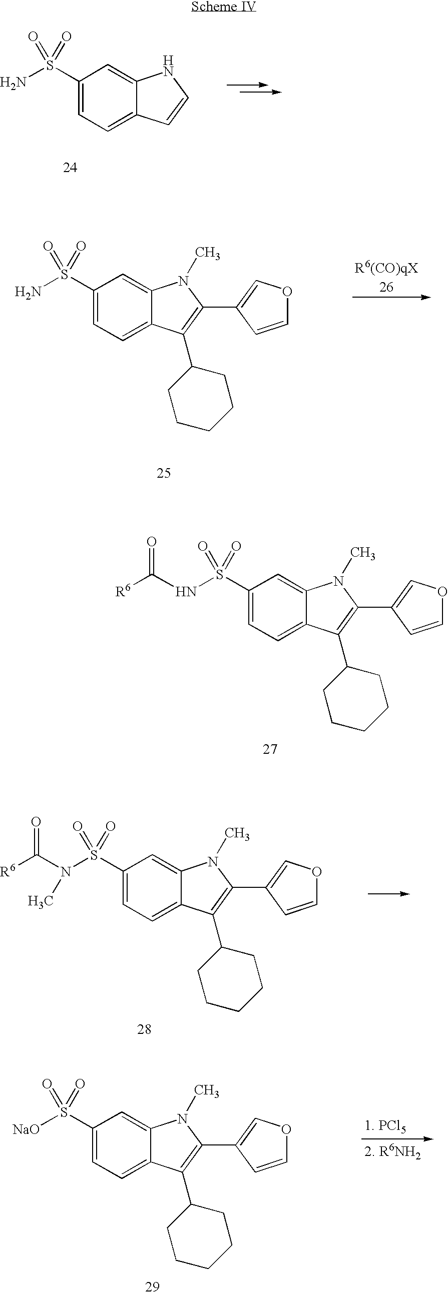

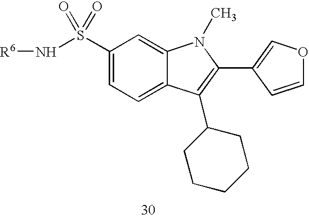

As shown in Scheme IV, replacement of starting material 6-nitroindole (17) with 6-indolesulfonamide (24) and proceeding in a manner similar to that shown in Scheme III above provides indole 25. The indolesulfonamide is acylated with an acid halide (26, R⁶ is alkyl) in the presence of a base to provide compound 27. The 6-indolesulfonamide is synthesized as described in U.S. Pat. No. 5,169,860. The acid derived from acid halide 26 can also be used as a coupling partner provided that it is activated by a peptide coupling reagent. The sulfonamide (27) can be alkylated in an inert solvent such as DMF with an alkyl halide such as iodomethane in the presence of a base to provide alkylated derivative 28, which in turn can be hydrolyzed by aqueous sodium hydroxide to afford the sodium salt of indolesulfonic acid 29.

Treatment of sulfonic acid 29 with a chlorinating reagent such as phosphorous pentachloride in an inert solvent provides the activated intermediate which upon treatment with hydroxylamine (R⁶ is hydroxy) in an inert solvent by methods well known to practitioners of the art provides compounds of formula 30.

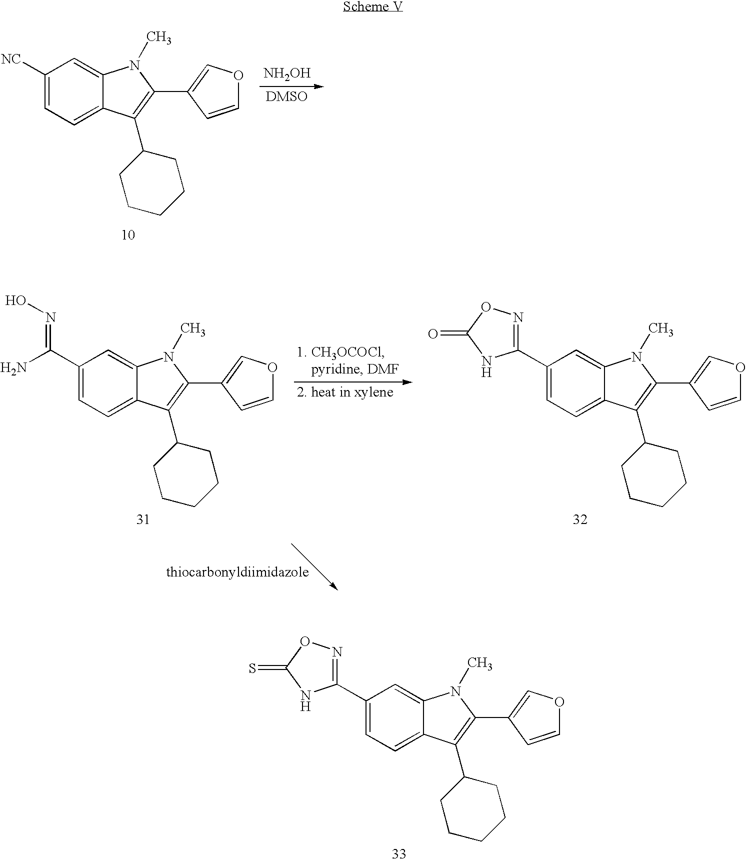

As shown in Scheme V, treatment of nitrile 10 with hydroxylamine in DMSO at about 70° C. affords the amidoxime 31 which upon treatment with methyl chloroformate, carbonyldiimidazole, or thiocarbonyldiimidazole affords the acidic heterocycles 32 and 33.

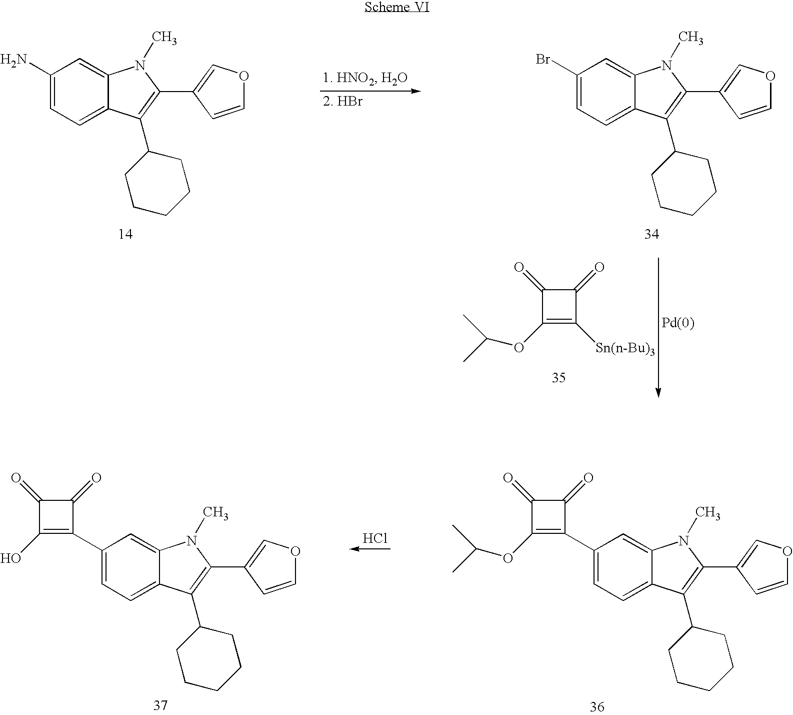

In Scheme VI, indole 14 is treated with nitrous acid in the presence of hydrobromic acid to afford bromide 34. Palladium-mediated cross-coupling of 34 with 3-isopropoxy-4-(tributylstannyl)-3-cyclobutene-1,2-dione (35) according to the method of Liebeskind and and Feng1 (Journal of Organic Chemistry, 55(19), 5359, 1990) gives squarate ester 36. Brief treatment with hydrochloric acid gives acidic heterocycle 37.

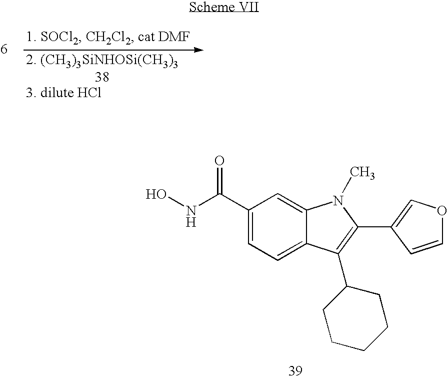

As shown in Scheme VII, hydroxamic acids are compounds which can be prepared within the scope of the present disclosure. For example, the hydroxamic acid 39 is synthesized as illustrated above. A solution of acid 6 in methylene chloride containing a catalytic amount of DMF is converted to the acid chloride by the action of thionyl chloride. Treatment of the solution with an excess of N,O-bis-trimethylsilylhydroxlamine (38) affords 39 after brief treatment with aqueous hydrochloric acid.

### EXAMPLES

The present invention will now be described in connection with certain preferred embodiments which are not intended to limit its scope. On the contrary, the present invention covers all alternatives, modifications, and equivalents as can be included within the scope of the claims. Thus, the following examples, which include specific embodiments, will illustrate one practice of the present invention, it being understood that the examples are for the purposes of illustration of certain embodiments and are presented to provide what is believed to be the most useful and readily understood description of its procedures and conceptual aspects.

The majority of the final compounds were purified by reverse phase chromatography using a preparative C-18 column employing gradients of methanol-water containing 0.1% of trifluoroacetic acid (TFA), and using a Shimadzu High Perfomance Liquid Preparative Chromatographic System employing an XTERRA 30×100 mm S5 column at 40 mL/min flow rate with a 12 min gradient. The final compounds usually precipitated from the aqueous eluent mixture when the methanol co-solvent was removed. An Emrys Optimizer personal microwave reactor was used for the microwave assisted reactions. Molecular weights and purities were usually determined using a Shimadzu LCMS. NMR spectra were usually obtained on either a Bruker 500 or 300 MHz instrument. The preparative silicic acid plates were 20×20 cm with a 1000 micron layer of silica gel GF.

### Example 1

### 3-Cyclohexyl-6-(5-tetrazolyl)-2-furan-3-yl-1-methyl-1H-indole (11)

A solution of oxalyl chloride in methylene chloride (0.17 μL 2.0 M, 0.34 mmol) was added to a stirred solution of 3-cyclohexyl-2-furan-3-yl-1-methyl-1H-indole-6-carboxylic acid (6) (100 mg, 0.023 mmol) in methylene chloride (3 mL). A drop of DMF was added and the solution heated to reflux, then stirred at ambient temperature for 30 minutes. The solution was cooled in an ice-water bath and methanolic ammonia (1.0 mL of 2.0 M, 2.0 mmol) rapidly added. The mixture was warmed to room temperature and was diluted with methylene chloride. The solution was washed (2×water, brine), dried (magnesium sulfate), filtered, and concentrated to provide 3-cyclohexyl-2-furan-3-yl-1-methyl-1H-indole-6-carboxamide (8) as an off-white solid: ESI-MS m/e 323 (MH+).

Phosphorous oxychloride (3 mL) was added in one portion to the amide 8 (100 mg). An exothermic reaction ensued (caution), and after 20 minutes the mixture was concentrated by rotary evaporation. Ice was added to the residue followed by an excess of cold dilute aqueous ammonium hydroxide. The solids were extracted with ethyl acetate, and the extract was washed (dilute aqueous ammonium hydroxide, brine), dried (sodium sulfate), filtered, and concentrated to provide crude 3-cyclohexyl-2-furan-3-yl-1-methyl-1H-indole-6-carbonitrile (10) as a dark solid. A solution of the nitrile in methylene chloride was applied to a silicic acid prep plate and eluted with methylene chloride. The appropriate band was extracted with methylene chloride (2×). Recrystallization from hexanes afforded 10 as a pale yellow solid: ESI-MS m/e 305 (MH+).

Azidotributyltin (150 μL, 0.55 mmol) was added to a solution of the nitrile 10 (12.5 mg, 0.041 mmol) in toluene (400 μL), and the solution was sonicated for 30 min at 160° C. The toluene was removed with a gentle stream of nitrogen, and the residue was dissolved in DMF and the desired tetrazole (9) isolated as a colorless solid after preparative reverse phase chromatography: ESI-MS m/e 348 (MH+).

### Example 2

### 3-Cyclohexyl-N-(5-tetrazolyl)-2-furan-3-yl-1-methyl-1H-indole-6-carboxamide (9)

A mixture of acid 6 (100 mg, 0.31 mmol), 5-aminotetrazole (32 mg, 0.31 mmol), TBTU (100 mg, 0.31 mmol), and TEA (156 mg, 1.55 mmol) in DMSO (350 μL) was stirred at 22° C. for 3 hours. The mixture was poured into cold water/acetic acid to precipitate 9, and a solution of the precipitate in hot 1.0 N sodium hydroxide (1.5 mL) was treated with decolorizing carbon and filtered. The filtrate was cooled, acidified with dilute hydrochloric acid, and the precipitated solid collected and crystallized from acetic acid to afford 9 as a buff solid: ESI-MS m/e 391 (MH+); ¹H NMR (300 MHz, DMSO) δ 1.23-1.87 (m, 2H), 1.69-1.96 (m, 8H), 2.73 (m, 1H), 3.69 (s, 3H), 6.76 (s, 1H), 7.77 (d, J=9.5 Hz, 1H), 7.87 (d, J=8.8 Hz, 1H), 7.92 (S, 1H), 7.99 (s, 1H), 8.40 (s, 1H), 12.16 (br. s, 1H).

### Example 3

### tert-Buyl 3-cyclohexyl-2-(furan-3-yl)-1-methyl-1H-indol-6-ylcarbamate (13)

Diphenylphosphoryl azide (0.12 mL 0.56 mmol) was added to a solution of 3-cyclohexyl-2-(furan-3-yl)-1H-indole-6-carboxylic acid (6) (150 mg, 2.46 mmol) and triethylamine (0.14 mL, 0.73 mmol) in tert-butyl alcohol (8 mL) in a microwave vial. The vial was sealed and heated at 140° C. for 15 minutes in a microwave apparatus. The mixture was dissolved in ethyl acetate. The solution was washed with brine and dried (sodium sulfate). The solution was filtered. Concentration of the filtrate provided crude 21(120 mg) as a yellow semi-solid. The crude product was purified on silicic acid with methylene chloride:ethyl acetate(8:1) to afford 13 as a colorless solid: ESI-MS m/e 394 (MH+).

### Example 4

### 3-Cyclohexyl-2-(furan-3-yl)-1-methyl-1H-indol-6-amine (14)

A solution of tert-buyl 3-cyclohexyl-2-(furan-3-yl)-1-methyl-1H-indol-6-ylcarbamate (13) (11 mg, 0.028 mmol) in methylene chloride (0.25 mL) and TFA (0.25) was stirred for 30 minutes at 22° C. The solution was concentrated to provide 14 as a greenish solid: ESI-MS m/e 294 (MH+).

### Compound Activity

The compounds listed in Table 2 below were tested for biological activity using the HCV RdRp cell line and FRET assay described below. The activity ranges were classified into the following groups: for IC₅₀, A (least active) >1 μM; B 0.1-1 μM; C (most active) <0.1 μM; for EC₅₀, D (least active) >10 μM; E 5-10 μM, F (most active) <5 μM. In one embodiment, the IC₅₀ values are from about 0.0001-10 μM. In another embodiment the values are from about 0.0001-1 μM and in another embodiment the values are less than about 0.1 μM. In one embodiment, the EC₅₀ values are from about 0.001 to 20 μM, in another embodiment from about 0.001-10 μM and in another embodiment less than about 5 μM.

TABLE 2
COMPOUND ACTIVITY

| | | RdRp Inhibition | |
|---|---|---|---|
| | | Range, μM | |
| Compound | Structure | IC₅₀ | EC₅₀ |
| 1 | 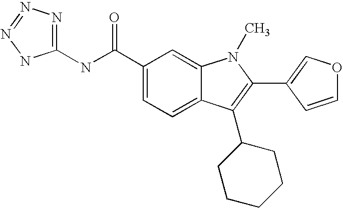 | B | F |
| 2 | 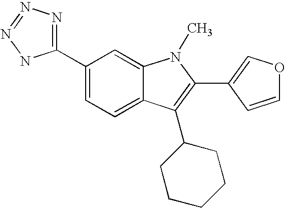 | B | E |

### Biological Studies

A HCV RdRp assay was utilized in the present disclosure, and was prepared, conducted and validated as follows:

HCV NS5B RdRp cloning, expression, and purification. The cDNA encoding the NS5B protein of HCV, genotype 1b, was cloned into the pET21a expression vector. The protein was expressed with an 18 amino acid C-terminal truncation to enhance the solubility. The E. coli competent cell line BL21(DE3) was used for expression of the protein. Cultures were grown at 37° C. for ˜4 hours until the cultures reached an optical density of 2.0 at 600 nm. The cultures were cooled to 20° C. and induced with 1 mM IPTG. Fresh ampicillin was added to a final concentration of 50 μg/ml and the cells were grown overnight at 20° C.

Cell pellets (3 L) were lysed for purification to yield 15-24 mgs of purified NS5B. The lysis buffer consisted of 20 mM Tris-HCl, pH 7.4, 500 mM NaCl, 0.5% triton X-100, 1 mM DTT, 1 mM EDTA, 20% glycerol, 0.5 mg/ml lysozyme, 10 mM MgCl₂, 15 ug/ml deoxyribonuclease I, and Complete TM protease inhibitor tablets (Roche). After addition of the lysis buffer, frozen cell pellets were resuspended using a tissue homogenizer. To reduce the viscosity of the sample, aliquots of the lysate were sonicated on ice using a microtip attached to a Branson sonicator. The sonicated lysate was centrifuged at 100,000×g for 1 hr at 4° C. and filtered through a 0.2 μm filter unit (Corning).

The protein was purified using three sequential chromatography steps: Heparin sepharose CL-6B, polyU sepharose 4B, and Hitrap SP sepharose (Pharmacia). The chromatography buffers were identical to the lysis buffer but contained no lysozyme, deoxyribonuclease I, MgCl₂ or protease inhibitor and the NaCl concentration of the buffer was adjusted according to the requirements for charging the protein onto the column. Each column was eluted with a NaCl gradient which varied in length from 5-50 column volumes depending on the column type. After the final chromatography step, the resulting purity of the enzyme is >90% based on SDS-PAGE analysis. The enzyme was aliquoted and stored at −80° C.

HCV NS5B RdRp enzyme assay. HCV RdRp genotype 1b assays were run in assay buffer composed of 20 mM Tris-HCl, pH 7.5, 2.5 mM KCl, 5 mM MgCl₂, 1 mM DTT, 1.6 U RNAse inhibitor (Promega N2515), in 96 well plates (Falcon 3918). All compounds were serially diluted in DMSO and diluted further in assay buffer such that the final concentration of DMSO in the assay was 2%. Compounds were serially diluted (3-fold each time) for a 7 point inhibition analysis. HCV RdRp genotype 1b enzyme was used at a final concentration of 28 nM. A polyA template was used at 28 nM, and the oligo-dT₁₂₋₁₈ primer was used at 840 nM final concentration. Preannealed primer and template were obtained commercially (Amersham 27-787802). ³H-UTP was used at 0.125 μCi (1 μM total UTP). Reaction was initiated by the addition of enzyme. Reactions were incubated at 30° C. for 45 min, and stopped by adding 30 ul of 20% ice cold TCA. Plates were chilled for 30 minutes and harvested onto Unifilter-96, GF/B plates(Packard, 6005177) using a Packard FilterMate Cell Harvester. The harvest plates were prewashed 3 times, 200 ul/well, with 100 mM NaPPi. Harvested filters were washed 30 times, 200 ul/well, with distilled water followed by ethanol. Filter plates were dried, and 30 ul/well microscint-20 was added. Plates were read on a Packard Top Count NXT.

The IC₅₀ values for compounds were determined using six different [I], combined with 7 nM enzyme, 800 ng of the template-primer polyC/oligoG₁₂ (1:5 molar ratio), and 0.7 uM of ³H GTP containing 1 uCi. The observed fractional activity (fa=vi/vo) was used in the equation IC₅₀=[I]/(1/fa−1) to determine a single point IC₅₀ value. Typically, the single point IC₅₀ values derived from [I] that produced fractional activities in the range of 0.1 to 0.8 relative to the uninhibited control were averaged to calculate the IC₅₀ value for each compound.

FRET Assay Preparation. To perform the HCV FRET screening assay, 96-well cell culture plates were used. The FRET peptide (Anaspec, Inc.) (Taliani et al., Anal. Biochem. 240:60-67 (1996), expressly incorporated by reference in its entirety) contains a fluorescence donor, EDANS, near one end of the peptide and an acceptor, DABCYL, near the other end. The fluorescence of the peptide is quenched by intermolecular resonance energy transfer (RET) between the donor and the acceptor, but as the NS3 protease cleaves the peptide the products are released from RET quenching and the fluorescence of the donor becomes apparent.

The assay reagent was made as follows: 5× cell Luciferase cell culture lysis reagent from Promega (#E153A) diluted to 1× with dH₂O, NaCl added to 150 mM final, the FRET peptide diluted to 20 uM final from a 2 mM stock. Cells were trypsinized, placed into each well of a 96-well plate and allowed to attach overnight. The next day, the test compounds were added to columns 1 through 10; column 11 was media only, and column 12 contained a titration of interferon as a control (1000 units for A12, B12, 100 units for C12, D12, 10 units for E12, F12 and 1 unit for G12, H12). The plates were then placed back in the incubator.

FRET Assay and Cytotoxicity Assay. Subsequent to addition of the test compounds described above (FRET Assay Preparation), at various times the plate was removed and Alamar blue solution (Trek Diagnostics, #00-100) was added per well as a measure of cellular toxicity. After reading in a Cytoflour 4000 instrument (PE Biosystems), plates were rinsed with PBS and then used for FRET assay by the addition of 30 ul of the FRET peptide assay reagent described above (FRET Assay Preparation) per well. The plate was then placed into the Cytoflour 4000 instrument which had been set to 340 excite/490 emission, automatic mode for 20 cycles and the plate read in a kinetic mode. Typically, the signal to noise using an endpoint analysis after the reads was at least three-fold.

Compound analysis was determined by quantification of the relative HCV replicon inhibition and the relative cytotoxicity values. To calculate cytoxicity values, the average Alamar Blue fluorescence signals from the control wells in row 11 were set as 100% non-toxic. The individual signals in each of the compound test wells were then divided by the average control signal and multiplied by 100% to determine percent cytotoxicity. To calculate the HCV replicon inhibition values, an average background value FRET signal was obtained from the two wells containing the highest amount of interferon at the end of the assay period. These numbers were similar to those obtained from naïve Huh-7 cells.

The background numbers were then subtracted from the average FRET signal obtained from the control wells in row 11 and this number was used as 100% activity. The individual signals in each of the compound test wells were then divided by the averaged control values after background subtraction and multiplied by 100% to determine percent activity. EC₅₀ values for an interferon titration were calculated as the concentration which caused a 50% reduction in HCV RNA, HCV protein amounts or FRET activity. The two numbers generated for the compound plate, percent cytoxicity and percent activity were used to determine compounds of interest for further analysis.

Although the disclosure has been described with respect to specific aspects, those skilled in the art will recognize that other aspects are intended to be included with the scope of the claims which follow.

1. A compound of formula (I) 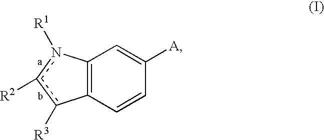
or a pharmaceutically acceptable salt thereof, wherein 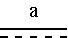
is a single or double bond; 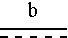
is a single or double bond;
provided that at least one of 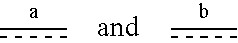
is a single bond;
A is selected from —NH₂, (NR^{a}R^{b})sulfonyl, an unsaturated 5-membered ring having 3 or 4 heteroatoms selected from nitrogen, oxygen, and sulfur, wherein the ring is optionally substituted with one or two substituents selected from oxo and (thio)oxo, 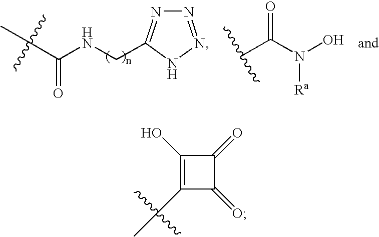
wherein n is 0 to 3 and 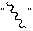
denotes the point of attachment of the substituent to the parent molecule;
R¹ is selected from hydrogen, alkenyl, alkyl, and alkynyl;
R² is selected from aryl and heteroaryl;
R³ is selected from cycloalkenyl and cycloalkyl;
R^{a} is selected from hydrogen, alkenyl, alkyl, and alkynyl; and
R^{b} is selected from hydroxy and alkylcarbonyl.

2. The compound of claim 1 wherein A is selected from —NH₂, (NR^{a}R^{b})sulfonyl,

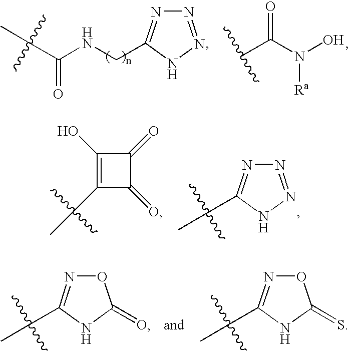

3. The compound of claim 1 wherein A is selected from

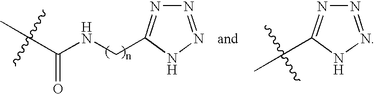

4. The compound of claim 3 wherein A is

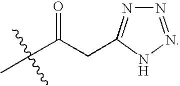

5. The compound of claim 3 wherein A is

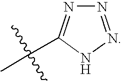

6. A compound of formula (II) 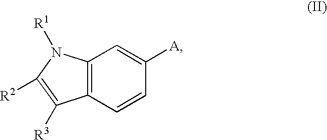
or a pharmaceutically acceptable salt thereof, wherein
A is selected from —NH₂, (NR^{a}R^{b})sulfonyl, 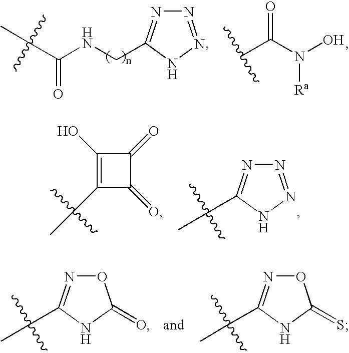
wherein n is 0 to 3 and 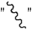
denotes the point of attachment of the substituent to the parent molecule;
R¹ is selected from hydrogen and alkyl;
R² is heteroaryl;
R³ is cycloalkyl;
R^{a} is selected from hydrogen, alkenyl, alkyl, and alkynyl; and
R^{b} is selected from hydroxy and alkylcarbonyl.

7. A compound of formula (III) 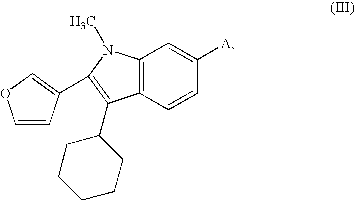
or a pharmaceutically acceptable salt thereof, wherein
A is selected from

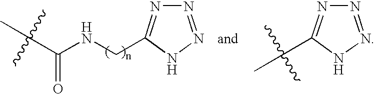

8. A composition comprising a compound of claim 1 and a pharmaceutically acceptable carrier.

9. The composition of claim 8 further comprising an interferon and ribavirin.

10. The composition of claim 8 further comprising another compound having anti-HCV activity.

11. The composition of claim 10 wherein the other compound having anti-HCV activity is an interferon.

12. The composition of claim 11 wherein the interferon is selected from interferon alpha 2B, pegylated interferon alpha, consensus interferon, interferon alpha 2A, and lymphoblastiod interferon tau.

13. The composition of claim 10 wherein the other compound having anti-HCV activity is selected from interleukin 2, interleukin 6, interleukin 12, a compound that enhances the development of a type 1 helper T cell response, interfering RNA, anti-sense RNA, Imiqimod, ribavirin, an inosine 5′-monophospate dehydrogenase inhibitor, amantadine, and rimantadine.

14. The composition of claim 10 wherein the other compound having anti-HCV activity is a small molecule compound.

15. The composition of claim 10 wherein the other compound having anti-HCV activity is effective to inhibit the function of a target selected from HCV metalloprotease, HCV serine protease, HCV polymerase, HCV helicase, HCV NS4B protein, HCV entry, HCV assembly, HCV egress, HCV NS5A protein, IMPDH and a nucleoside analog for the treatment of an HCV infection.

16. A method of inhibiting the function of the HCV NS5B protein comprising contacting the HCV NS5B protein with the compound of claim 1.

17. A method of treating an HCV infection in a patient, comprising administering to the patient a therapeutically effective amount of the compound of claim 1 or a pharmaceutically acceptable salt thereof.

18. The method of claim 17 wherein the compound is effective to inhibit the function of the HCV NS5B protein.

19. The method of claim 17 further comprising administering another compound having anti-HCV activity prior to, after, or simultaneously with a compound of claim 1.

20. The method of claim 19 wherein the other compound having anti-HCV activity is an interferon.

21. The method of claim 20 wherein the interferon is selected from interferon alpha 2B, pegylated interferon alpha, consensus interferon, interferon alpha 2A, and lymphoblastiod interferon tau.

22. The method of claim 19 wherein the other compound having anti-HCV activity is selected from interleukin 2, interleukin 6, interleukin 12, a compound that enhances the development of a type 1 helper T cell response, interfering RNA, anti-sense RNA, Imiqimod, ribavirin, an inosine 5′-monophospate dehydrogenase inhibitor, amantadine, and rimantadine.

23. The method according of claim 19 wherein the other compound having anti-HCV activity is a small molecule.

24. The method of claim 23 wherein the other compound having anti-HCV activity is effective to inhibit the function of a target selected from HCV metalloprotease, HCV serine protease, HCV polymerase, HCV helicase, HCV NS4B protein, HCV entry, HCV assembly, HCV egress, HCV NS5A protein, IMPDH and a nucleoside analog for the treatment of an HCV infection.

25. The method of claim 23 wherein the other compound having anti-HCV activity is effective to inhibit the function of a target in the HCV life cycle other than the HCV NS5B protein.

26. Use of the compound of claim 1 for the manufacture of a medicament for treating HCV infection in a patient.

27. Use of the composition of claim 8 for the manufacture of a medicament for treating HCV infection in a patient.
